(19) United States
(12) Patent Application Publication
(10) Pub. No.: US 2005/0032188 A1
(43) Pub. Date: Feb. 10, 2005

(54) Construction of bacillus licheniformis T1 strain, and fermentation production of crude enzyme extract therefrom

(76) Inventor: Jason C. H. Shih, Cary, NC (US)

Correspondence Address: MYERS BIGEL SIBLEY & SAJOVEC, PO BOX 37428, RALEIGH, NC, 27627, US

(21) Appl. No.: 10/661,172

(22) Filed: Sep. 13, 2003

Related U.S. Application Data
(60) Provisional application No. 60/410,710, filed on Sep. 13, 2002.

(51) Int. Cl. C12N 9/54; C12N 1/21

(52) U.S. Cl. 435/221; 435/252.31

(57) ABSTRACT

A recombinant bacteria and methods of making and using the same are provided. The recombinant bacteria is a recombinant Bacillus having at least one heterologous kerA coding segment inserted into the chromosome thereof, with the recombinant Bacillus producing greater quantitites of keratinase than a corresponding wild-type Bacillus that does not have the at least one heterologous kerA coding segment inserted into the genome thereof. The Bacillus may be Bacillus licheniformis or Bacillus subtilis, and the the kerA coding segment may be a Bacillus licheniformis or Bacillus subtilis kerA coding segment.

### RELATED APPLICATIONS

This application claims the benefit of U.S. provisional patent application Ser. No. 60/410,710, filed Sep. 13, 2002, the disclosure of which is incorporated by reference herein in its entirety.

### FIELD OF THE INVENTION

The present invention relates to construction a recombinant Bacillus licheniformis T399D strain (“hereinafter T1 strain”), and fermentation production of, more specifically scale-up production of, crude enzyme extract containing keratinase by using such recombinant Bacillus licheniformis T1 strain.

### DESCRIPTION OF THE RELATED ART

Keratinase, which is a serine protease specifically able to degrade keratin protein in poultry feathers, has been successfully produced and isolated from a feather-degrading bacterium Bacillus licheniformis PWD-1. In addition to promoting the hydrolysis of feather keratin, the keratinase is capable of hydrolyzing a broad spectrum of protein substrates, including casein, collagen, elastin, etc., and it displays higher proteolytic activity than most other proteases known in the art.

One important potential commercial application of keratinase, among many others, is the use of the crude dried cell-free fermentation product from keratinase-producing B. licheniformis strains as a feed additive to supplement poultry feed, in a manner that improves the digestibility and nutritional value of such feed.

However, a major problem in commercializing keratinase is the high production cost of such enzyme.

Thus, two approaches have been taken to solve this problem,: (1) strain development to develop bacterial strains with improved keratinase production; and (2) process development to design efficient production strategies for fermentation and extraction of the keratinase enzyme.

It is therefore an object of the present invention to provide recombinant Bacillus licheniformis strains that overproduce keratinase and demonstrate significantly higher enzyme yield than that of the wild-type Bacillus licheniformis PWD-1 strain.

It is another object of the present invention to provide methods for commercially feasible mass production of keratinase enzyme that suits the application of such crude fermentation product as a feed additive and the destruction of infectious prions, and purifed fermentation product for biomedical research applications.

### SUMMARY OF THE INVENTION

A first aspect of the present invention is a recombinant Bacillus having at least one heterologous kerA coding segment inserted into the chromosome thereof, with the recombinant Bacillus producing greater quantitites of keratinase than a corresponding wild-type Bacillus that does not have the at least one heterologous kerA coding segment inserted into the genome thereof. The Bacillus may be Bacillus licheniformis or Bacillus subtilis, and the the kerA coding segment may be a Bacillus licheniformis or Bacillus subtilis kerA coding segment. The corresponding wild-type Bacillus is Bacillus licheniformis PWD-1. In a preferred embodiment the recombinant Bacillus has a plurality of the heterologous kerA coding segment inserted into the chromosome thereof, and in a particularly preferred embodiment has from 3 to 5 of the heterologous kerA coding segment inserted into the chromosome thereof. In a preferred embodiment the recombinant Bacillus is a protease-deficient Bacillus. The kerA coding segment is operatively associated with promoter, preferably a constitutive promoter such as a P43 promoter.

A second aspect of the invention is a bacterial culture comprising a recombinant Bacillus as described herein in a culture media. The culture media preferably comprises not more than 3% protein substrate, and in a particularly preferred embodiment the culture media comprises 1% soy and 1% feather meal.

A third aspect of the present invention is a method of making a recombinant Bacillus as described herein, comprising the steps of: (a) inserting a kerA coding segment into an integrative Bacillus expression vector, the kerA coding segment operatively associated with a promoter, the promoter operative in Bacillus bacteria; and then (b) transforming a Bacillus with the integrative Bacillus expression vector. Preferably the integrative Bacillus expression vector includes alpha-amylase 5′- and 3′-flanking DNA segments, and the kerA coding segment is inserted between the alpha amylase 5′- and 3′-flanking segments. Particularly preferred is a pLAT10 vector.

A fourth aspect of the present invention is a method of making a keratinase, comprising: (a) culturing a recombinant Bacillus as described herei in a media; and then (b) collecting the keratinase from the media. Preferably the media comprises not more than 3% protein substrate, and in a particularly preferred embodiment the media comprises 1% soy and 1% feather meal.

The foregoing and other objects and aspects of the present invention are explained in greater detail in the drawings herein and the specification set forth below.

### BRIEF DESCRIPTION OF THE DRAWINGS

FIG. 1. Isolation of kerA from Bacillus licheniformis PWD-1.

FIG. 2. Effect of medium on keratinase production from the new transformant PJT-3. Protease activity was determined by the azocasein assay.

### DETAILED DESCRIPTION OF THE INVENTION AND PREFERRED EMBODIMENTS THEREOF

The present invention can be practiced based upon the disclosure described herein, in light of the knowledge of persons skilled in the art, and in light of the information set forth in the U.S. Pat. No. 5,712,147, U.S. Pat. No. 5,525,229, U.S. Pat. No. 5,186,961, U.S. Pat. No. 5,171,682, U.S. Pat. No. 5,063,161, U.S. Pat. No. 4,959,311, U.S. Pat. No. 4,908,220, US Patent Application No. 20030108991 (Titled “Immobilization of Keratinase for Proteolysis and Keratinolysis); US Patent Application No. 20020192731 (titled “Method and Composition for Sterilizing Surgical Instruments”) and US Patent Application No. 20020172989 (titled “Composition and Method for Destruction of Infectious Prion Proteins”), the disclosures of all of which are incorporated by reference herein in their entirety.

Construction of Recombinant Bacillus licheniformis T399D Strains. For developing better bacterial strains that can overproduce the keratinase enzyme, two approaches have been used to overproduce the enzyme: 1) increasing the gene copy number in Bacillus via a plasmid-containing strain or 2) generating multiple gene copies in the chromosome of the bacterial strain.

The kerA gene has been cloned and expressed from B. subtilis (Lin, X., S. L Wong, E. S. Miller, and J. C. H. Shih. (1997), Expression of the Bacillus licheniformis PWD-1 keratinase gene in B. subtilis, J. Ind. Microb. Biotech, 19: 134-138) and E. coli (Wang, J. J. and J. C. H. Shih (1999), Fermentation production of keratinase from Bacillus licheniformis PWD-1 and a recombinant B. subtilis, J. Ind. Microb. Biotech. 22: 608-616). However, plasmid-based enzyme expression in Bacillus was not stable because of the segregational instability during fermentation. Formation of inclusion bodies and complicated in vitro refolding of pro-keratinase presented a challenge for keratinase expression in an E. coli system and resulted in limited enzyme yield. Although chromosomal integration has frequently been applied to improve gene expression, instability of tandemly amplified chromosomal genes has been reported (Albertini, A. M. and A. Galizzi (1985), Amplification of chromosomal region in Bacillus subtilis, J. Bacteriol. 163: 1203-1211; Young, M. (1984), Gene amplification in Bacillus subtilis, J. Gen. Microbiol. 130: 1913-1921).

The present invention constructs an integrative vector that carries the kerA gene, and then transforms and integrates such vector into the protease-deficient asporogenic host strain B. licheniformis T399D. Through a single crossover Campbell recombination, multiply-integrated copies of the kerA gene are introduced into the chromosome of B. licheniformis T399D. The resulting recombinant B. licheniformis T399D strain demonstrates a significantly increased enzyme production rate compared to that of the wild-type B. licheniformis PWD-1 strain.

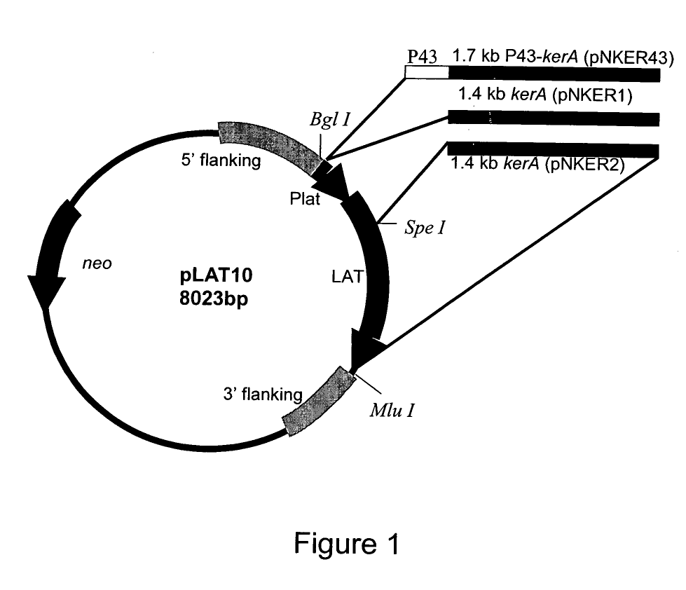

Bacillus licheniformis PWD-1 (ATCC 53575) was used in the present invention to isolate the kerA gene, as shown in FIG. 1. B. licheniformis T399D (provided from DSM, NV, Het Overloon 1, 6411 T E Heerlen, The Netherlands, and described in the following patent references: PCT Wo85/0038; PCT WO88/0662; PCT WO91/1315; EP 0572088; EP 0635574) was used as host for cloning and expression studies. The plasmid pLB29, carrying the P43 promoter promoter (Wang and Doi, 1987) and kerA, was used as the gene source for cloning. An integrative Bacillus expression vector pLAT10, derived from pLAT8 (provided from DSM, NV, Netherlands), containing α-amylase 5′ and 3′ flanking regions was used to facilitate integration of the whole vector into the host chromosome. PWD-1 was grown in feather, soy, or Luria-Bertani (LB) medium at 50° C. The B. licheniformis T399D strain was grown at 37° C. in LB medium containing 20-50 μg/mL neomycin for routine transformation and gene expression.

DNA Manipulation. Plasmids from Bacillus were prepared by the rapid alkaline sodium dodecyl sulfate method (Rodriguez and Tait, 1983). Chromosomal DNA of PWD-1 was isolated using the method described by Doi (1983). Restriction enzymes and DNA ligases were purchased from Promega and Boehringer-Mannheim and were used as recommended by the manufacturers. PCR was performed with either Pfu (Boehringer-Mannheim) or Taq (Promega) DNA polymerase under the following conditions: 94° C. for 1 min, 56° C. for 1.5 min, 72° C. for 2 min (30 cycles) and 72° C. for 5 min. DNA fragments were separated by 0.8 to 1.2% agarose gel. The desired DNA fragment and PCR products were recovered and purified by the QIAquick Gel Extraction Kit and QIAquick PCR Purification Kit (Qiagen Inc, Calif.), respectively.

Gene Cloning, Transformation and Integration in B. licheniformis DB104. he kerA (1.4 kb) and P43-kerA (1.7 kb) were amplified by PCR from pLB29 plasmid using the primers as described in Table 1, as follows:

TABLE 1
PCR PRIMERS FOR SUBCLONING THE kerA GENE INTO pLAT10

| Primer | Sequence (5′ → 3′) |
|---|---|
| Bgl I Upper | GAGTAAGAGCCATATCGGCCAAGCTGAAGCGGTCTATTCATAC |
| | (SEQ ID NO: 1) |
| Spe I Upper | AGTAAGAACTAGTCAAGCTGAAGCGGTCTATTCATAC |
| | (SEQ ID NO: 2) |
| Mlu 1 Lower | GGAACGGACGCGTAATATTGGACAACCTTCATCAGAATG |
| | (SEQ ID NO: 3) |
| P43-Bgl-5′ | GTCTGTAGCCATATCGGCGAATTCGAGCTCAGCATTATTGAGTGG |
| | (SEQ ID NO: 4) |
| KERA3 | ATTTAAATTATTCTGAATAAAGAGG |
| | (SEQ ID NO: 5) |
| KERA4 | CACTAGCTTTTTCTATATGCTATTTG |
| | (SEQ ID NO: 6) |

An amplified DNA fragment containing kerA or P43-kerA was ligated into the vector between the α-amylase 5′- and 3′-flanking DNA sequences of digested plasmid, replacing all of the α-amylase DNA sequence, as shown in FIG. 1.

Newly constructed plasmids (pNKER1, PNKER2 and pNKER43) described above were further transformed into B. subtilis DB104. Transformation of B. subtilis DB104 was carried out by the competence cell method as previously described (Lin et. al, 1997). The fidelity of the kerA insert in vectors was verified by restriction enzyme digestion analysis.

After growing the positive transformants in LB medium containing 20 mg/L neomycin, the keratinase activity was detected from transformants pNKER1/DB104, PNKER2/DB104 and pNKER43/DB104, as shown in Table 2:

TABLE 2
Keratinase expression from B. subtilis DB104

| Plasmid | Promoters/vector | Milk agar plate | Azocasein, U/mL |
|---|---|---|---|
| pNKER1 | Pker/pLAT10 | + | 2600 |
| pNKER2 | Plat-Pker/pLAT10 | + | 2613 |
| pNKER43 | P43-Pker/pLAT10 | +++ | 5200 |
| pLB29 | P43-Pker/pUB18 | +++ | 4920 |
| pLAT10 | — | — | 40 |

*All strains were grown in LB medium at 37° C. for 24 hr.

Transformation, integration and expression in B. licheniformis T399D. The newly constructed integration plasmids pNKER1 and pNKER43 were isolated from B. licheniformis DB104 and transformed into B. licheniformis T399D by the modified protoplast method (Sanders et al. 1997; van der Lann et al, 1991). All possible transformant candidates were further confirmed for the gene insertion by restriction digestion and PCR amplification. Integration occurred by single crossover Campbell recombination; the complete plasmid integrated into either the complementary 5′- or 3′-α-amylase flanking region of the host chromosome. The final stable copy number achieved was approximately determined by Southern Blot analysis.

Screening and Stabilization of Transformants. Transformants from regeneration agar plates were grown on milk agar plates at 37° C. overnight. New clones producing keratinase based on halo formation were inoculated into LB medium containing different levels of neomycin (10-100 μg/mL) as a selection marker. After growth in LB medium at 37° C. overnight, the culture was incubated at 45° C. for 4-6 hours to cure the free plasmid. Subsequently, the stabilization procedure was carried out by transferring these transformants to a nonselective 1% soy medium and incubated at 37° C. for 2 days. The culture supernatant was analyzed for protease activity by the azocasein/azokeratin assay. The candidates for strains over-expressing keratinase were further transferred to fresh nonselective media for at least seven generations to confirm the stability of new strains.

More than 500 positive transformants (based on halo formation on milk agar plates) were screened on both solid and liquid medium containing various levels of neomycin (0 to 100 ug/mL). After more than ten generations, eighteen (PJT1 to PJT18, as shown in Table 3 below) T399D transformants were selected based on keratinase yield:

TABLE 3
Screening of transformants over-expression of keratinase

| ^{a}Strain | ^{b}Enzyme activity, U/mL | Relative, % |
|---|---|---|
| PWD-1 | 2360 | 100 |
| PJT-1 | 5440 | 231 |
| PJT-2 | 4560 | 193 |
| PJT-3 | 5860 | 248 |
| PJT-4 | 5300 | 225 |
| PJT-5 | 6420 | 272 |
| PJT-6 | 4420 | 187 |
| PJT-7 | 4960 | 210 |
| PJT-8 | 5560 | 236 |
| PJT-9 | 4380 | 186 |
| PJT-10 | 4680 | 198 |
| PJT-11 | 4666 | 198 |
| PJT-12 | 4280 | 181 |
| PJT-13 | 4460 | 189 |
| PJT-14 | 3360 | 142 |
| PJT-15 | 4380 | 186 |
| PJT-16 | 3138 | 133 |
| PJT-17 | 3540 | 150 |
| PJT-18 | 3180 | 135 |

^{1.}All strains were grown in 1% soy medium at 37° C.
^{2.}Enzyme activity was determined by azocasein assay.

Colony PCR was used to identify integration of the kerA gene in these transformants. All selected strains contained the 1.4 kb kerA gene and no free plasmids were detected in the cell.

As compared to wild type B. licheniformis PWD-1 at the same growth conditions, the protease activity produced from these new transformants was increased up to 2.7-fold. The keratinase yield from three transformants (PJT16, PJT3 and PJT4) was further analyzed by Western blot (data not shown).

The result indicated that the protease expressed from new clones could be specifically probed by anti-keratinase antiserum. After quantification of enzyme expression by measuring the gel band density, the keratinase produced from PJT16, PJT3, and PJT4 was enhanced by 1.6, 2.9, and 2.1-fold, respectively.

Gene and protein analysis. The integration gene copy number of transformed DNA was analyzed by Southern hybridization techniques (Sambrook et al, 1989). Total isolated chromosome DNA was isolated and digested with restriction enzymes. After electrophoresis, the DNA was transferred onto a nitrocellulose membrane (Sigma). Digoxigenin-labeled probes for the detection of kerA gene were amplified from pLB 29 by PCR using the PCR DIG Labeling mix (Boehringer-Mannheim, Mannheim, Germany). Hybridization was carried out at 42° C. in a hybridization oven, using a hybridization buffer as recommended by the manufacturer.

The culture media of transformants were collected and assayed for proteolytic and keratinolytic activities (Lin et al., 1992). Precipitated by 5% TCA, concentrated proteins were analyzed by sodium dodecyl sulfate polyacrylamide gel electrophoresis SDS-PAGE (Laemmli, 1970). Western blotting was modified as described by Towbin et al. (1979). From SDS-PAGE, proteins were transferred to a nitrocellulose membrane and probed with anti-keratinase rabbit antiserum.

The quantitation of DNA and protein concentrations from Southern and Western Blots was performed by using Chemilmager™ 4400 gel documentation system and PhaEase™ Image Analysis software (Alpha Innotech Corp, CA)

Keratinase activity was measured by azokeratin hydrolysis as described previously (Lin et al.,1992). Hydrolysis of azocasein was modified and used to determine the total protease activity (Sarath et al., 1989). The protein concentration was determined by the Bio-Rad Microassay procedure (Bradford, 1976).

Protein expression from multiple chromosomal integration. Multiple gene chromosomal integration was confirmed by Southern blot analysis. Five new strains were chosen for analysis. The results indicated that enzyme production was enhanced by integrating multiple gene copies into the chromosome, but protein secretion was not linearly proportional to gene copy number. Strains with greater than six integrated copies of the kerA gene demonstrated decreased enzyme yield. The optimal number for increased expression of keratinase was 3-5 gene copies in the chromosome.

Constitutive promoter P43 and medium effects on keratinase production. The constitutive promoter P43, when cloned in front of kerA, resulted in improved keratinase expression in T399D, as shown in Table 4:

TABLE 4
Keratinase yield enhanced by P43 promoter

| ^{a}Strain | plasmid/Host | ^{b}Keratinase activity, U/mL | Activity, % |
|---|---|---|---|
| PWD-1 | — | 450 | 100 |
| PJT1 | pNKER43/T399D | 2480 | 551 |
| PJT2 | pNKER43/T399D | 2449 | 544 |
| PJT3 | pNKER43/T399D | 2794 | 620 |
| PJT6 | pNKER43/T399D | 2041 | 453 |
| PWN21 | pNKER1/T399D | 259 | 65 |
| PWN315 | pNKER1/T399D | 236 | 52 |
| PWN523 | pNKER1/T399D | 133 | 29 |
| PWN-627 | pNKER1/T399D | 23 | 5 |
| PWN-339 | pNKER1/T399D | 358 | 79 |

¹All strains were grown in 1% soy and 1% FM medium at 37° C.
²Keratinase activity was measured by azokeratin assay.

When the P43 promoter was excluded from the expression vector, the keratinase expression levels dropped below that of PWD-1. All positive clones transformed from pNKER without the P43 promoter have lower keratinase yields than PWD-1. PWN339, the best clone, only produced 80% of the enzyme activity of PWD-1, even though this clone contained multiple gene copies. This result demonstrated that the P43 promoter significantly improves the transcription efficiency of kerA in both B. subtilis and B. licheniformis.

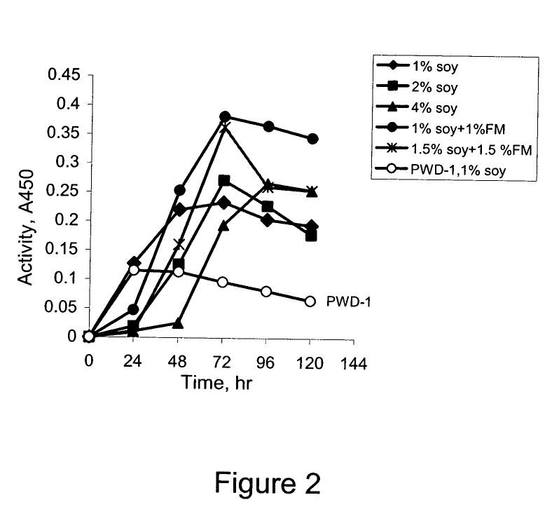

In order to characterize the media effects on keratinase production from isolated integrants, higher concentrations of substrates were used. As shown in FIG. 2, total protease activity was increased when higher concentrations of soy or feather meal were included in the fermentation media. The enzyme yield was found to drop when more than 3% protein substrate was used. The optimal media condition contained 1% soy mixed with 1% feather meal—in this media keratinase activity increased about four-fold compared to PWD-1.

In the present invention, stable B. licheniformis strains carrying multiple integrated kerA in chromosome were constructed to overproduce keratinase. Different gene copy number ranging from one to eight (data not shown) in the chromosome was successfully isolated by incorporating certain degrees of neomycin in the selective medium. Compared to the B. subtilis expression system, stable integrants producing higher enzyme activity were developed. Unlike the plasmid-containing expression system in B. subtilis, the chromosomal integration of kerA in B. licheniformis avoided the segregational and structural instability common to replicative plasmids (Bron and Luxen, 1985; Harington et al., 1988; Primrose and Ehrlich, 1981).

It was also demonstrated that multiple gene copies in the chromosome above a certain copy number (data not shown) was detrimental to higher production of keratinase. transformants with about 16 gene copies in the chromosome demonstrated lower keratinase activity than lower-copy number strains. Strains with copy numbers of 3 to 5 per chromosome were shown to be optimal for keratinase production.

When the P43 promoter was introduced into the expression cassette and integrated into strain T399D, the keratinase yield was significantly increased compared to integrants with the native promoter only. These results indicated that this strong promoter was useful for improving the transcriptional efficiency and played an important role for the expression of keratinase from T399D.

The new strains could grow on medium containing up to 3% soy or feather meal and demonstrated a doubling of enzyme activity in this media (as shown in FIG. 2). In contrast, when PWD-1 was grown in the same media at levels higher than 2% soy or feather meal, the enzyme production was repressed (Wang and Shih, 1999). This result facilitated the use of higher concentrations of protein substrate in the media to improve keratinase production in large-scale fermentation.

In summary, new strains with multiple copies of kerA integrated into the chromosome of B. licheniformis T399D were developed. Gene copy numbers and expression in integrants were determined by Southern and Western Blot, respectively. When the transformed strains were grown under the media conditions of 1% soy and 1% feather meal (FM), keratinase activity was increased about 4-6 fold (as shown in FIG. 2).

Fermentation Production of Crude Keratinase Enzyme Using Recombinant Bacillus licheniformis T399D strain. A fermentation scale-up strategy was designed for the production of keratinase, using the recombinant Bacillus licheniformis T399D strain (hereinafter the “Bacillus licheniformis TI strain”).

Flask Culture in LB Medium. Flask culture was carried out in Luria-Bertani (LB) medium that was prepared according to the manufacturer's specification, containing: 1.0L of distilled water, 15 g Bacto agar, 10 g NaCl, 10 g Bacto tryptone, and 5.0 g yeast extract. Bacillus licheniformis strain T1 was streaked from glycerol stock onto LB plates and grown at 37° C. for 18 hours. A single colony of Bacillus licheniformis Ti was then transferred from the LB plate into a flask that contained 500 ml LB medium, and grown at 37° C. for 6 hours, while the cell growth was monitored by measuring the optical density at 660 nm, (Beckman DU Series 660 Spectrophotometer, Fullerton, Calif.). After 6 hours of growth, the OD₆₆₀ measured above 1.0.

Seed Cultures. Seed cultures for Bacillus licheniformis TI strain were grown in a medium containing: 0.7 g/L KH₂PO₄, 1.4 g/L K₂HPO₄, 0.1 g/L MgSO₄.7H₂O, 10 g/L defatted NUTRISOY® soy flour (from Archer Daniels Midland Co., Decatur, Ill.), and 0.1 g/L Antifoam 204 or 289 (from Sigma Chemical Co., St. Louis, Mo.). The initial seed culture pH was adjusted to 7.0, by adding 1M HCl or NaOH.

The 500 ml flask culture was transferred into a first stage seed fermentor of about 10L to 20L that contained the seed culture medium, and was grown therein at 37° C. for 8 hours to reach 2.5% to 5% inoculum size. The first stage seed culture was then transferred to a second stage seed fermentor of 100L, 250L or 800L, and was grown therein at 37° C. for 8 hours.

Production Media. The production culture medium used for Bacillus licheniformis Ti strain contains 0.7 g/L KH₂PO₄, 1.4 g/L K₂HPO₄, 0.1 g/L MgSO₄.7H₂O, 13 g/L defatted NUTRISOY® soy flour (from Archer Daniels Midland Co., Decatur, Ill., USA), 40 g/L Lodex5 (commercialized as C*dry MD01960 from Cerestar USA, Hammond, Ind.), 13 g/L feather meal, and 0.1 g/L Antifoam 204 or 289 (from Sigma Chemical Co., St. Louis, Mo., USA). The initial production culture pH was adjusted to 7.0, by adding 1M HCl or NaOH.

The second stage seed culture was transferred to a production fermentor that contained the production culture medium for final stage culturing. The final stage culture was carried out at 37° C. for 26 hours, reaching a total culturing time of 48 hours before harvesting.

During the above culturing steps, the initial pH of the culture medium was adjusted to 7.0, but no pH control was provided. The optimal level of dissolved oxygen is about 30% for Bacillus licheniformis Ti strain. The inoculum size was about 2.5 to 5%, and the inoculum age was about 12 hours.

Recovery and Downstream Processing. The enzyme activity in the production culture was checked before harvesting. The culture supernatant was separated from the cell mass via centrifuge, and then concentrated via ultrafiltration or evaporation. The concentrate liquid enzyme was then spray-dried.

Alternatively, the culture supernatant was directly spray-dried after separation from the cell mass, without being concentrated.

Enzyme Yield and Enzyme Activity. For 100 L production culture, the enzyme activity measured by azocasein assay before harvesting was 30,000 to 35,000 U/mL, and the cell number was 6×10⁹ CFU/mL. The total dry weight of the 100 L production culture was 40 g/L, including 15 g/L insoluble dry weight and 25 g/L soluble dry weight.

The crude enzyme yield from the directly dried culture supernatant is 20 g/L, while the crude enzyme yield form a culture concentrate, as obtained via Pellicon filtration with 10 kDa molecular weight cut, was 16 g/L. The enzyme activity of the crude dry enzyme was more than 1,000,000 U/g, as measured by azocasein assay.

The crude dry keratinase enzyme extract produced according to the method described hereinabove can be supplemented in poultry feed as a feed additive, in a manner that improves the digestibility and nutritional value of such feed.

### REFERENCES

Albert, B., H. Tjalsma, H. E. Smith, A. dejong, R. Meima, G. Venema, S. Bron, and J. M. van Dijl. 1999. Evaluation of Bottlenecks in the late stages of protein secretion on Bacillus subtilis. Appl. Env. Microb. 65: 2934-2941.
Albertini, A. M. and A. Galizzi. 1985. Amplification of chromosomal region in Bacillus subtilis. J. Bacteriol. 163: 1203-1211.
Bolhuis, A., A. Sorokin, V. Azevedo, S. D. Ehrlich, P. G. Braun, A. de Jong, G. Venema, S. Bron, and J. M. van Dijl. 1996. Bacillus subtillis can modulate its capacity and specificity for protein secretion through temporally controlled expression of the sips gene for signal peptidase I. Mol. Microbiol. 22:605˜518.
Bradford M. 1976. A rapid and sensitive method for the quantitation of microgram quantities of protein utilizing the principle of protein-dye binding. Anal. Biochem. 72: 248-254.
Bron, S. and E. Luxen. 1985. Segregational instability of pUB 110 derived recombinants in Bacillus subtilis. Plasmid. 14:234-244.
de Boer, A. S., F. Priest, and B. Diderichsen. 1994. On the industrial use of Bacillus licheniformis: a review. Applied Microbiol. Biotechnol. 40: 595-598.
Diderichsen B, Poulsen G B, Jorgensen P L. 1991 Cloning and expression of an amylase gene from Bacillus stearothermophilus. Res Microbiol. 142:793-796
Doi, R. H. 1983. Isolation of Bacillus subtilis chromosomal DNA. P.162-163. In R. L. Rodriquez and R. C. Trait(ed.), Recombinant DNA techniques. Addison-Wesley Publishing Co., Inc., Reading, Mass.
Driessen, A. J. M. 1994. How proteins cross the bacterial cytoplasmic membrane. J Membr. Biol. 142:145-159.
Harington, A. T. G. Watson. M. E. Louw, J. E. Rodel, and J. A. Thomson. 1988. Stability during fermentation of a recombinant α-amylase plasmid in Bacillus subtilis. Appl. Microbiol. Biotechnol. 27: 521-527.
Kawamura, F., and R. H. Doi. 1984. Construction of a Bacillus subtilis double mutant deficient in extracellular alkaline and neural protease. J. Bacteriol. 160:442-444.
Kontinen, V. P., and M. Sarvas. 1993. The PrsA protein is essential for protein secretion in Bacillu subtilis and sets a limit for high-level secretion. Mol. Microbiol. 8:727-737.
Laemmli, K. 1970. Cleavage of structure proteins during the assembly of the head of bacteriophage T4. Nature. 227: 680-685.
Lin, X., C. G. Lee, E. S. Casale, and J. C. H. Shih. 1992. Purification and characterization of a keratinase from a feather-degrading Bacillus licheniformis strain. Appl. Env. Microb. 58: 3271-3275.
Lin, X., S. L Wong, E. S. Miller, and J. C. H. Shih. 1997. Expression of the Bacillus licheniformis PWD-1 keratinase gene in B. subtilis, J Ind. Microb. Biotech, 19: 134-138.
Priest F G, and C R Harwood. 1994. Bacillus species., p. 377-421. In. Y. H. Hui and G G Khachatourians (ed), Food Biotechnology, VCH Publishers Inc, New York.
Primrose, S. B. and S. D. Ehrlich. 1981. Isolation of plasmid deletion mutants and study of their instability. Plasmid. 6: 193-200.
Pugsley, A. P. 1993. The complete general secretory pathway in gram negative bacteria. Microbiol. Rev. 57:50-108.
Rodriquez, R. L. and R. C. Trait. 1983. Recombinant DNA techniques. Addison-Wesley Publishing Co., Inc., Reading, Mass.
Sambrook, J. E. F Fritsch, and T. Maniatis. 1989. Molecular cloning: A Laboratory Manual, 2^{nd} Ed. Cold Spring Harbor Laboratory Press, Cold Spring Harbor, N.Y.
Sanders, J. P. M., J. A. van den Berg, P. M. Andreoli, B. Kortrijk, Y. J. Vos, J. H. van Ee, L. J. S. M. Mulleners. 1997. Transformed industrial Bacillus strains and methods for making and using them. U.S. Patent. 5624829.
Sarath G., R. D. Motte, and F. W. Wagner. 1989. Protease assay methods. p. 25-55. In R. J. Beynon and J. S. Bond (ed.). Proteolytic enzymes: a practical approach. IRL Press, Oxford.
Towbin, H. T., T. Staehelin, and J. Gordon. 1979. Electrophoretic transfer of proteins from polyacrylamide gels to nitrocellulose sheet: Procedure and some applications. Proc. Natl. Acad. Sci. USA 76:4350-4354.
van der Laan J C, Gerritse G, Mulleners L J, van der Hoek R A, Quax W J. 1991. Cloning, characterization, and multiple chromosomal integration of a Bacillus alkaline protease gene. Appl Environ Microbiol. 57:901-909.
von Heijne, G. 1990. The signal peptide. J. Membr. Biol. 115:195-201.
Wang, J. J. and J. C. H. Shih. 1999. Fermentation production of keratinase from Bacillus licheniformis PWD-1 and a recombinant B. subtilis. J. Ind. Microb. Biotech. 22:608-616.
Wang, L. F. and R. H. Doi. 1987. Promoter switch during development and termination site of the Υ⁴³ operon of Bacillus subtilis. Mol. Gen. Genet. 207: 114-119.
Wu, S. C., R. Ye, X. C. Wu, S. C. Ng, and S. L. Wong. 1998. Enhanced secretory production of a single-chain antibody fragment from Bacillus subtilis by coproduction of molecular chaperones. J. Bacteriol. 180:2830-2835.
Wu, X. C, W. Lee, L. Tran, and S. L Wong. 1991. Engineering a Bacillus subtilis expression-secretion system with a strain deficient in a six extracellular proteases. J. Bacteriol. 173: 4952-4958.
Young, M. 1984. Gene amplification in Bacillus subtilis. J. Gen. Microbiol. 130: 1913-1921.

Although the invention has been described with respect to various illustrative embodiments, features and aspects, it will be appreciated that the utility of the invention is not thus limited, but rather extends to and includes various other modifications, alterations and other embodiments, as will readily suggest themselves to those of ordinary skill in the art based on the disclosure herein. The invention is therefore intended to be broadly construed, as encompassing all such modifications, alterations and other embodiments within the spirit and scope of the ensuing claims.

1. A method of making a keratinase, comprising:
(a) culturing a recombinant Bacillus in a media, said recombinant Bacillus having at least one heterologous kerA coding segment inserted into the chromosome thereof, with said recombinant Bacillus producing greater quantities of keratinase than a corresponding wild-type Bacillus that does not have said at least one heterologous kerA coding segment inserted into the genome thereof, and then
(b) collecting said keratinase from said media.

2. The method of claim 1, wherein said media comprises not more than 3% protein substrate.

3. The method of claim 1, wherein said media comprises 1% soy and 1% feather meal.

4. The method of claim 1, wherein said Bacillus is selected from the group consisting of Bacillus licheniformis and Bacillus subtilis.

5. The method of claim 1, wherein said Bacillus is Bacillus licheniformis.

6. The method of claim 1, wherein said kerA coding segment is a Bacillus licheniformis or Bacillus subtilis kerA coding segment.

7. The method of claim 1, wherein said kerA coding segment is a Bacillus licheniformis kerA coding segment.

8. The method of claim 1, wherein said corresponding wild-type Bacillus is Bacillus licheniformis PWD-1.

9. The method of claim 1, said recombinant Bacillus having a plurality of said heterologous kerA coding segment inserted into the chromosome thereof.

10. The method of claim 1, said recombinant Bacillus having from 3 to 5 of said beterologous kerA coding segment inserted into the chromosome thereof.

11. The method of claim 1, wherein said recombinant Bacillus is a protease-deficient Bacillus.

12. The method of claim 1, wherein said kerA coding segment is operatively associated with a constitutive promoter.

13. The method of claim 1, wherein said kerA coding segment is operatively associated with a P43 promoter.

14. A recombinant Bacillus having at least one heterologous kerA coding segment inserted into the chromosome thereof, with said recombinant Bacillus producing greater quantitites of keratinase than a corresponding wild-type Bacillus that does not have said at least one heterologous kerA coding segment inserted into the genome thereof.

15. The recombinant Bacillus of claim 14, wherein said Bacillus is selected from the group consisting of Bacillus licheniformis and Bacillus subtilis.

16. The recombinant Bacillus of claim 14, wherein said Bacillus is Bacillus licheniformis.

17. The recombinant Bacillus of claim 14, wherein said kerA coding segment is a Bacillus licheniformis or Bacillus subtilis kerA coding segment.

18. The recombinant Bacillus of claim 14, wherein said kerA coding segment is a Bacillus licheniformis kerA coding segment.

19. The recombinant Bacillus of claim 14, wherein said corresponding wild-type Bacillus is Bacillus licheniformis PWD-1.

20. The recombinant Bacillus of claim 14 having a plurality of said heterologous kerA coding segment inserted into the chromosome thereof.

21. The recombinant Bacillus of claim 14 having from 3 to 5 of said heterologous kerA coding segment inserted into the chromosome thereof.

22. The recombinant Bacillus of claim 14, wherein said recombinant Bacillus is a protease-deficient Bacillus.

23. The recombinant Bacillus of claim 14, wherein said kerA coding segment is operatively associated with a constitutive promoter.

24. The recombinant Bacillus of claim 14, wherein said kerA coding segment is operatively associated with a P43 promoter.

25. A bacterial culture comprising a recombinant Bacillus of claim 14 in a culture media.

26. The bacterial culture of claim 25, wherein said culture media comprises not more than 3% protein substrate.

27. The bacterial culture of claim 25, wherein said culture media comprises 1% soy and 1% feather meal.

28. A method of making a recombinant Bacillus of claim 14, comprising the steps of:
(a) inserting a kerA coding segment into an integrative Bacillus expression vector, said kerA operatively associated with a promoter, said promoter operative in Bacillus bacteria; and then
(b) transforming a Bacillus with said integrative Bacillus expression vector.

29. The method of claim 28, wherein said integrative Bacillus expression vector includes alpha-amylase 5′- and 3′-flanking DNA segments, and wherein said kerA coding segment is inserted between said alpha amylase 5′- and 3′-flanking segments.

30. The method of claim 28, wherein said integrative Bacillus expression vector is pLAT10.
